(19) United States
(12) Patent Application Publication
(10) Pub. No.: US 2023/0148650 A1
(43) Pub. Date: May 18, 2023

(54) NOVEL COATING COMPOSITION AND COATED COMPOSITIONS COMPRISING THE SAME

(71) Applicant: DSM IP ASSETS B.V., Heerlen (NL)

(72) Inventors: Elger FUNDA, Kaiseraugst (CH); Odile KRAINZ, Kaiseraugst (CH); Simone KÖNIG-GRILLO, Kaiseraugst (CH); Zdravka MISIC, Kaiseraugst (CH); Markus NOWOTNY, Kaiseraugst (CH); Loni SCHWEIKERT, Kaiseraugst (CH)

(21) Appl. No.: 18/099,831

(22) Filed: Jan. 20, 2023

Related U.S. Application Data
(63) Continuation of application No. 16/078,648, filed on Aug. 22, 2018, now abandoned.

(30) Foreign Application Priority Data
Feb. 26, 2016 (WO) 16157690.5

(51) Int. Cl. A23P 20/12; A23L 33/12; A23K 40/30; A23K 20/158; A23L 33/00; A23L 5/00; A23L 33/10; A23K 20/163; A23P 10/30; A23L 33/125; A23D 9/05; A61K 9/50; A61K 31/202

(52) U.S. Cl. CPC A23P 20/12; A23L 33/12; A23K 40/30; A23K 20/158; A23L 33/00; A23L 5/00; A23L 33/10; A23K 20/163; A23P 10/30; A23L 33/125; A23D 9/05; A61K 9/5036; A61K 31/202; A23V 2002/00

(57) ABSTRACT

Novel coating systems for coating solid particles which comprise PUFAs (and/or salts thereof) are provided wherein the coating system includes at least one cyclodextrin. Compositions coated with such a coating system and the use of such compositions in the production of food, feed, dietary supplements and/or pharmaceutical products are also described.

### CROSS-REFERENCE TO RELATED APPLICATIONS

This application is a continuation of U.S. application Ser. No. 16/078,648 filed on Aug. 22, 2018 (now abandoned), which in turn is the U.S. national phase of International Application No. PCT/EP2017/053860 filed 21 Feb. 2017, which designated the U.S. and claims priority to EP Patent Application No. 16157690.5 filed 26 Feb. 2016, the entire contents of each of which are hereby incorporated by reference.

### FIELD

The present patent application relates to a novel coating composition for coating solid particles, which comprise PUFAs (and/or salts thereof), wherein the coating composition comprises at least one cyclodextrin. Furthermore the present application relates to coated compositions coated with such a coating compositions and the use of such coated compositions in the production of food, feed, dietary supplements and/or pharmaceutical products.

### BACKGROUND AND SUMMARY

Poly-unsaturated fatty acids (PUFAs) as well as their salts (such the Na, K or Ca salts) are very well known compounds for a healthy diet (especially the omega-3 fatty acids). The PUFAs (especially the omega-3 fatty acids) have a variety of health benefits such as i.e. health benefits against cardiovascular diseases (CVDs) including well-established hypotriglyceridemic and anti-inflammatory effects.

PUFAs can be found in a variety of plants and animals. A very good source of omega-3 fatty acids are i.e. fish.

Alternatively, PUFAs can also be produced synthetically.

Due to the fact that many consumers do not like fish, it is very common to add PUFAs (and/or salts thereof) to other dietary products (enrich these products with PUFA).

The problem with the PUFAs as well as with their salts is, that they have strong tendency to oxidise. This results in a loss of the PUFAs in the product and secondly (even worse) in the development of a strong and very unpleasant smell. With an increasing number of double bonds, the PUFAs are subject to increasing oxidative degradation and development of undesirable “off-flavors”, mainly fishy and rancid smell and taste.

Volatile degradation products cause off-flavor even at very low concentration. Sensory properties of a product may become unacceptable even before a loss of PUFAs can be analytically detected.

Stabilization of PUFAs by formation of inclusion complexes is a well-known technique for encapsulation of hydrophobic substances. However, as a delivery form for PUFAs these inclusion complexes have some serious drawbacks. Due to the fact that inclusion complexes are formed in a molar 1:1 ratio, the formation of inclusion complexes limits the maximum payload of the obtained powder. It is also assumed that in case of PUFA triglycerides only one PUFA chain enters the cavity of cyclodextrin, leaving the others unprotected such that they can easily be oxidised. Finally, the main disadvantage of encapsulated PUFA forms is not the loss of PUFA oxidation but rather the formation of very small amounts of volatile degradation products, leading to fishy, painty or rancid smell of the product. As Hadaruga et al (Hadaruga, Daniel I.; Unlusayin, Mustafa; Gruia, Alexandra T.; Birau, Cristina; Rusu, Gerlinde; Hadaruga, Nicoleta G.,Beilstein Journal of Organic Chemistry (2016), 12, 179-191, doi: 10.3762/bjoc.12.20) have shown, inclusion complexes with cyclodextrin can reduce but not completely prevent PUFA oxidation and therefore the problem of the small amounts of volatile degradation products is not solved by encapsulating PUFA in cyclodextrin inclusion complexes.

A goal of the present invention was to provide a powderous formulation with high content of PUFA (usually more than 10 weight-% (wt-%), based on the total weight of the powderous formulation), which has significantly improved stability in regard to the development of the undesirable “off-flavors.

In other words, the powderous formulation having a high content of PUFA (and/or salts thereof) according to the present invention does not smell (fishy) even after storage.

Surprisingly it was found that when a coating composition, which comprises at least 10 wt-% of at least one cyclodextrin, based on the total weight of the coating composition, is used, the so coated solid particles comprising PUFA (and/or salts thereof) have no (or a significantly reduced) tendency of developing undesirable “off-flavors”.

We have found that a beneficial effect on sensory stability of PUFA formulations can be achieved, when a powder containing PUFA is coated with a layer of cyclodextrin. In this case, cyclodextrin does not form an inclusion complex with PUFA but rather with the above mentioned small amounts of volatile degradation products. Since no molar 1:1 ratio with PUFA is required, much higher payloads can be achieved.

Therefore the present invention relates to a coating composition (CS) comprising at least 10 wt-% of cyclodextrin, based on the total weight of the coating composition.

### BRIEF DESCRIPTION OF THE DRAWINGS

FIG. 1 diagrammatically shows the concept of the present invention which compares the coated compositions according to the embodiments of the present invention with the prior art embodiments.

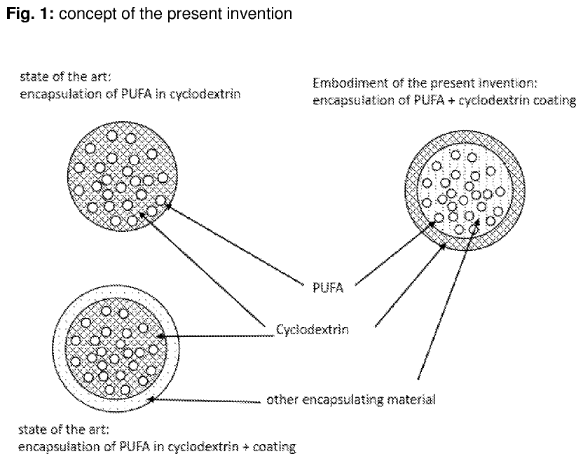

### DETAILED DESCRIPTION OF EMBODIMENTS

Cyclodextrins (sometimes called cycloamyloses) are a family of compounds made up of sugar molecules bound together in a ring (cyclic oligosaccharides). Cyclodextrins are produced from starch by means of enzymatic conversion. They are composed of 5 or more α-D-glucopyranoside units linked 1→4, as in amylose (a fragment of starch). The 5-membered macrocycle is not natural. Recently, the largest well-characterized cyclodextrin contains 32 1,4-anhydroglucopyranoside units, while as a poorly characterized mixture, at least 150-membered cyclic oligosaccharides are also known.

Typical cyclodextrins contain a number of glucose monomers ranging from six to eight units in a ring, creating a cone shape. They can be classified into three groups:

α (alpha)-cyclodextrin: 6-membered sugar ring molecule

β (beta)-cyclodextrin: 7-membered sugar ring molecule

γ (gamma)-cyclodextrin: 8-membered sugar ring molecule

The coating composition can be pure cyclodextrin (which can be one single cyclodextrin or a mixture of a αcyclodextrin and/or β-cyclodextrin: and/or γ-cyclodextrin).

Therefore the present invention relates to a coating composition (CS1), which is coating composition (CS) consisting of cyclodextrin.

Therefore the present invention relates to a coating composition (CS1′), which is coating composition (CS1) consisting of one single cyclodextrin.

Therefore the present invention relates to a coating composition (CS1″), which is coating composition (CS1) consisting of a mixture of α-cyclodextrin and/or β-cyclodextrin: and/or γ-cyclodextrin.

Therefore the present invention relates to a coating composition (CS2), which is coating composition (CS) comprising one single cyclodextrin.

Therefore the present invention relates to a coating composition (CS2′), which is coating composition (CS) comprising of a mixture of a αcyclodextrin and/or β-cyclodextrin: and/or γ-cyclodextrin.

The coating composition can comprise further auxiliary compounds, such as binders, forming compounds (such as hydrocolloids, which can be either a polysaccharide or a protein), plasticizers (such as sugars like sucrose or a sugar derivative (mannitol, sorbitol), glycerol, mono- and diglyceride, acetylated monoglyceride, polyethylene glycol (PEG), polypropylene glycol), fillers, dyestuffs, flavors, antioxidants, etc.

These ingredients can be used in an amount of up to 90 wt-%, based on the total weight of the coating composition.

Therefore the present invention relates to a coating composition (CS3), which is coating composition (CS), (CS2) or (CS2′) comprising at least one auxiliary compound (up to 90 wt-%, based on the total weight of the coating composition, of at least one auxiliary compound).

Therefore the present invention relates to a coating composition (CS3′), which is coating composition (CS3), wherein the at least one auxiliary compound is chosen from the group consisting of binders, forming compounds (such as hydrocolloids, which can be either a polysaccharide or a protein), plasticizers (such as sugars like sucrose or a sugar derivative (mannitol, sorbitol), glycerol, mono- and diglyceride, acetylated monoglyceride, polyethylene glycol (PEG), polypropylene glycol), fillers, dyestuffs, flavors and antioxidants.

Preferred plasticizers according to the present invention are sugars like sucrose or a sugar derivative (mannitol, sorbitol), glycerol, mono- and diglyceride, acetylated monoglyceride, polyethylene glycol (PEG), polypropylene glycol. Preferably the PEG has a molecular weight between 200 and 6000 g/mol. It is clear that one single plasticizer can be used as well as mixtures of two and more plasticizers.

Therefore the present invention relates to a coating composition (CS3″), which is coating composition (CS3) or (CS3′) comprising at least one plasticizer chosen from the group consisting of sucrose or a sugar derivative (mannitol, sorbitol), glycerol, mono- and diglyceride, acetylated monoglyceride, polyethylene glycol (PEG), polypropylene glycol (preferably the PEG has a molecular weight between 200 and 6000 g/mol).

The coating composition according to the present invention is used for coating a (particulate) solid formulation comprising PUFAs (and/or salts thereof). Such a coated composition comprises a core (comprising the PUFA or a mixture of various PUFAs) and the coating composition.

PUFAs are classified according to the position of the double bonds in the carbon chain of the molecule as n-9, n-6 or n-3 PUFAs. Examples of n-6 PUFAs are linoleic acid (C18:2), arachidonic acid (C20:4), γ-linolenic acid (GLA, C18:13) and dihomo-γ-linolenic acid (DGLA, C20:3). Examples of n-3 PUFAs are α-linolenic acid (C18:13), eicosapentaenoic acid (EPA, C20:5), and docosahexaenoic acid (DHA, C22:6). Especially EPA and DHA have attracted interest of the food industry in recent years. The most available sources of these two fatty acids are fish and the marine oils extracted from them. Suitable PUFA salts are the sodium, potassium or calcium salts.

Therefore the present invention also relates to a coated composition (CC) comprising
(a) a core, which is a solid formulation comprises at least one PUFA (and/or salts thereof) and
(b) a coating composition, comprising
   (i) at least one cyclodextrin.

The PUFA is not encapsulated in the cyclodextrin.

Therefore the present invention also relates to a coated composition (CC1), which is coated composition (CC), wherein the at least one PUFA is chosen from the group consisting of n-9, n-6 or n-3 PUFAs and/or the salts thereof (especially the sodium, potassium or calcium salts).

Therefore the present invention also relates to a coated composition (CC1′), which is coated composition (CC) of (CC1), wherein the at least one PUFA is chosen from the group consisting of linoleic acid (C18:2), arachidonic acid (C20:4), γ-linolenic acid (GLA, C18:13), dihomo-γ-linolenic acid (DGLA, C20:3), α-linolenic acid (C18:13), eicosapentaenoic acid (EPA, C20:5) and docosahexaenoic acid (DHA, C22:6) and/or the salts thereof (especially the sodium, potassium or calcium salts).

All the preferences for the coating composition (b) apply to the above mentioned coated compositions.

In addition the core can comprise further ingredients, usually additives, which are used in the production of such compounds or additives which are useful for products in which the compositions according to the present invention are incorporated. The core of the composition can be in any form. For example, the core can be in the form of beadlet comprising the active ingredient. A suitable beadlet, which can be coated by the coating composition according to the present invention can be found in WO 2007/045488 (US 2012/0039964).

Furthermore the present invention relates to a coated composition (CC2), which is coated composition (CC), (CC1) or (CC1′) comprising
(i) 70 to 99.5 wt-%, based on the total weight of the coated composition, of a core and
(ii) 0.5 to 30 wt-%, based on the total weight of the coated composition, of a coating composition.

The core usually contains at least 10 wt-% of PUFA (and/or salts thereof), based on the total weight of the core; preferably at least 15 wt-% up to 50 wt-%.

Therefore the present invention relates to a coated composition (CC3), which is coated composition (CC), (CC1), (CC1′) or (CC2), wherein the core comprises at least 10 wt-% of PUFA (and/or salts thereof), based on the total weight of the core.

Therefore the present invention relates to a coated composition (CC3′), which is coated composition (CC), (CC1), (CC1′) or (CC2), wherein the core comprises 15 wt-% up to 50 wt-% of PUFA (and/or salts thereof), based on the total weight of the core.

The shape of the core as well as of the coated particles is also not an essential feature of the present invention. The shape can be sphere-like or any other form (also mixtures of shapes). Usually and preferably the particles are sphere-like.

The coating composition according to the present invention is layered around the core. Usually (and ideally) the coating covers the whole surface of the particle. Furthermore, the layer is usually (and ideally) equally thick on the surface of the core.

All the preferences for the coating composition apply to the above mentioned coated compositions.

The size of the core as well as the size of the coated particle is not an essential feature of the present invention. The coated particles are usually of such a size that tablets can be compacted.

A suitable size is between 50-1000 μm (preferably 100-800 μm); the size is defined by the diameter of the longest dimension of the particle and measured by commonly known method (like laser diffraction).

Therefore the present invention relates to a coated composition (CC4), which is coated composition (CC), (CC1), (CC1′), (CC2), (CC3) or (CC3′), wherein the average articles size of the coated particles is between 50 and 1000 μm.

Therefore the present invention relates to a coated composition (CC4′), which is coated composition (CC), (CC1), (CC1′), (CC2), (CC3) or (CC3′), wherein the average articles size of the coated particles is between 100 and 800 μm.

All particle sizes are determined by laser diffraction technique using a “Mastersizer 3000” of Malvern Instruments Ltd., UK. Further information on this particle size characterization method can e.g. be found in “Basic principles of particle size analytics”, Dr. Alan Rawle, Malvern Instruments Limited, Enigma Business Part, Grovewood Road, Malvern, Worcestershire, WR14 1XZ, UK and the “Manual of Malvern particle size analyzer”. Particular reference is made to the user manual number MAN 0096, Issue 1.0, November 1994. If nothing else is stated all particle sizes referring are Dv90 values (volume diameter, 90% of the population resides below this point, and 10% resides above this point) determined by laser diffraction. The particle size can be determined in the dry form.

The coated compositions (CC), (CC1), (CC1′), (CC2), (CC3), (CC3′), (CC4) and (CC4′) can be produced according to very well-known processes, such as spay drying, drum drying, spray granulation, agglomeration or beadlet processes. Generally, the coated particles are produced according to the following steps:
(i) the core (which comprises PUFA) is produced by commonly known and widely used processes;
(ii) the cores (solid particles) are coated in a second step by a solution, dispersion or slurry of the coating composition (cyclodextrin). The solution, dispersion or slurry is usually made by dissolving or suspending the coating material in water or an aqueous solution. Other suitable solvents like alcohol-water mixture may be used as well. The coating composition can be applied by well-known technologies such as fluid-bed coating or Wurster coating; and thereafter
(iii) the coated particles are dried and can be used for further purposes.

The coated compositions (CC), (CC1), (CC1′), (CC2), (CC3), (CC3′), (CC4) and (CC4′) of the invention can be used in any kind of formulations where the use of such fat soluble ingredients is useful. Usually the coated compositions can be used in food products, feed products, dietary supplements and/or pharmaceutical products.

Therefore the present invention relates to the use of coated compositions (CC), (CC1), (CC1′), (CC2), (CC3), (CC3′), (CC4) and (CC4′) in the production of food products, feed products, dietary supplements and/or pharmaceutical products.

Therefore the present invention relates to the production of food products, feed products, dietary supplements and/or pharmaceutical products, wherein coated compositions (CC), (CC1), (CC1′), (CC2), (CC3), (CC3′), (CC4) and (CC4′) are used.

The food products, feed products, dietary supplements and/or pharmaceutical products can be in any form (liquid, gel-like or solid).

As described above, these coated compositions do not “smell” fishy after storage.

The coated compositions according to the present invention can also be used as or used in dietary supplements. The dietary supplements can be in any form.

The coated compositions according to the present invention can also be used in pharmaceutical products. The pharmaceutical products can be in any galenical form, usually in the form of tablets.

A further embodiment of the present invention relates to food products, feed products, dietary supplements and/or pharmaceutical products, comprising at least one coated composition (CC), (CC1), (CC1′), (CC2), (CC3), (CC3′), (CC4) and/or (CC4′).

### EXAMPLES

The invention is illustrated by the following Example. All temperatures are given in ° C. and all parts and percentages are related to the weight.

### Example 1

An aqueous solution containing 4% beta cyclodextrin is produced by dissolving/dispersing 9 g beta cyclodextrin in 216 g water.

150 g beadlets containing ca 400 mg/g PUFA (mainly DHA and EPA) are fluidized in a lab-scale fluid-bed processor using Wurster technology. The cyclodextrin solution is sprayed on the fluidized particles at a product temperature of 55-64° C. After spraying, the product is dried in the processor at 49-59° C., A free flowing powder of coated beadlets is obtained. The product has a neutral smell, no fishy or rancid smell can be observed.

1. A particulate composition comprising coated particles, wherein each of the coated particles is comprised of:
(a) a particulate core formed of a solid formulation comprised of at least 15 wt. % up to 50 wt. %, based on total weight of the particulate cores, of at least one polyunsaturated fatty acid (PUFA) and/or a salt thereof, and
(b) a layer comprised of a coating composition coated around the solid formulation of the particulate core, wherein the coating composition comprises at least 10 wt. %, based on total weight of the coating composition, of cyclodextrin, wherein
the PUFA of the solid formulation is not encapsulated in the cyclodextrin and thereby the cyclodextrin does not form an inclusion complex with the PUFA.

2. The particulate coated composition according to claim 1, wherein
(a) the particulate cores of the coated particles are present in an amount of 70 to 99.5 wt. %, based on the total weight of the particulate composition, and
(b) the coating system of the coated particles is present in an amount of 0.5 to 30 wt. %, based on the total weight of the particulate composition.

3. The particulate coated composition according to claim 1, wherein the cyclodextrin and PUFA are present in the coated particles at a molar ratio which is not 1:1.

4. A process for producing the particulate composition according claim 1,
wherein the process comprises the sequential steps of:
(i) forming solid particulate cores comprised of the PUFA and/or a salt thereof by a beadlet process, a spray drying process or a spray-granulation process;
(ii) coating the solid particulate cores by a solution, dispersion or slurry coating process to provide a layer of the coating composition on the solid particulate cores to thereby form the coated particles; and thereafter
(iii) drying the coated particles to thereby form the particulate composition.

5. Food products, feed products, dietary supplements and/or pharmaceutical products comprising the particulate composition according to claim 1.

6. A coating system comprising at least 10 wt %, based on the total weight of the coating system, of cyclodextrin.
